(19) United States
(12) Patent Application Publication
(10) Pub. No.: US 2008/0077160 A1
(43) Pub. Date: Mar. 27, 2008

(54) Pain free electrical waxing hairs remover

(76) Inventor: Hoangmy Thi Nhan, Garden Grove, CA (US)

Correspondence Address: Hoangmy Thi Nhan, 13335 Newland St., Garden Grove, CA, 92844, US

(21) Appl. No.: 11/527,166

(22) Filed: Sep. 27, 2006

(51) Int. Cl. A61B 17/50

(52) U.S. Cl. 606/133

(57) ABSTRACT

This invention of innovative device is about helping people to remove their unwanted hairs.

This device would electrically wax away body and legs hairs effectively and economically. This device will remove body and legs hairs in a large numbers in much lesser time. Unlike chemical waxing or shaving to remove hair, this device has a new and distinguish concept of removing hairs. The hairs do not get waxed or shaved away. Instead, the hairs get unplugged by massaged off. This new concept will protect skin from cut, rash, irritated and burned and easily get the skin back to its normal healthy life.

This device also helps consumers to avoid chemical polluted and wasted. The best feature of all, that there is no hair waxing remover can provide, is “This device will wax away a larger number of hairs in lesser time with out skin pain”.

This new invention of Pain Free Hairs Remover has a scale down model. It is a much smaller size, which fit to remove hairs at any tight area of body like under arms, mustache and beard . . . .

This new invention is worth to be called a truly health and beauty aids personal care item.

### BACKGROUND

1. Field of Invention

Our human bodies create hairs for certain purposes. However, the living condition has been improving so much that make some parts of hairs, some time become less important and unwanted. The definition of a beautiful woman now has been including of certain part of body and legs to be hairs free. Hairs shaving and waxing equipments and products have become the big business of today.

Having been in the beauty business for seventeen years, I must see that waxing to remove hairs in general is the better and potent method over shaving. In waxing, the hairs take longer time to re-grow. How ever, waxing to remove hair is more expensive, more chemical pollution and wasted. Besides, chemical waxing to remove hairs might cause skin rash or irritation in some cases. There is one thing for sure, chemical waxing to remove hair certainly comes with pain, more or less individually.

I am, a naturalized American citizen woman proudly inventing “The Pain Free Electrical Waxing Hairs Remover”.

Unlike chemical waxing to remove hair, this device will have no chemical involved, no skin rash nor irritation. Although, this device will remove hairs in a larger quantity with much lesser time and virtually pain free. One must wonder of this amazing discovery to be in questionable.

2. Background Description of Art

Consumer can realize that the market now is full of hairs cutting equipments and hairs shaving products. They all provide us human the convenience way of life. Our daily shaving can remove hairs so quickly. The today technology has improved the sharpness of razor to its best top quality ever.

Chemical waxing hair remover has just come to the market lately. How ever, its successful business is much more than one can imagine.

Shaving can remove hairs so quickly. It needs time of preparing and cleaning up though. There is some time a little chance of cutting accident. The hairs will inevitable grow back next morning. In contrast, chemical waxing hairs remover has no chance of cutting accident, total removing hair down to its root, keeping the hair from re-grow in a much longer time. However, there is down side of using chemical waxing hair remover. There is good chance of skin rash, burned and certainly uncomfortable pain for hours.

The roots of hairs are connected with a very sensitive nerves system. Therefore, the skin is in pain when the hair is unplugged. That nerves system how ever, can be manipulated avoided recognizing the pain as the hair gets unplugged so quickly. Chemical waxing removes hair as quick as possible to avoid skin pain. The force to remove hair is a chemical adhesive agent that bonds to the hair. This agent is also bonded to skin that causes the skin's pain, burned and irritation in some case there after, when hair gets removed,

This device will provide electrical waxing to remove hair in a total difference concept of hair removal. In stead of quickly removing hair, this device allows its mechanical tweezers component to hold the hair in a hanging position. A quickly striking force will press down the skin to unplug the hair as the same time to manipulate the nerves system. The quick action force, that strikes on the skin controls the pain and removes the hair. There is virtually no pain at all there after match of hairs removed. The device at this design has 4 pairs of hairs tweezing holding and skin's striking elements. The more pairs of tweezing elements are the faster of the hairs to be removed.

### SUMMARY

This device has an electric motor that will drive the motion of tweezing hairs units and as the same instance strike the skin with its massage elements. The tweezing elements will open and close to catch hair, while the massage element, will be pressed down, strikes onto the skin of the hairs to vibrate manipulating the skin's nerves system.

The feature of this pain free hairs waxing remover is begin with its design. The device, while hairs of are holding at a hanging position, provides a striking force pressed down and vibrated the skin to get its nerves system manipulated. This device works as to massage skin while removing its hair. These coordinated actions of holding hairs and striking its skin will get the hairs come off from their skin easily and pain free. The skin, being massaged will get better blood circulation to become normal quickly after its hair was waxing away.

### OBJECTS, ADVANTAGE AND DESCRIPTIONS

### Objects

The Pain Free Electrical Waxing Hairs Remover will be chosen by consumers over other same category of products on the current market.
This new invented of electrical waxing hairs remover device will be used by a large number of consumers men or women.
If the unwanted hair is still existing then so is the Pain Free Electrical Waxing Hairs Remover.

### Advantages

This new invention of electrical waxing hair's remover device will save a lot of time, money, chemical and fabric wasted.
The size of device is so compact that will fit into any personal compartment.
The device is lightweight and without any hazardous material.
The device can wax away a large number of hairs in minutes with minimal time.
The device has a smaller scale down model that fit to remove hairs on any limited area of body.
The device has no cause of dangerous, no cut, no skin rash, no burned and no irritation.
The device will give consumers the most comfort and convenience in removing their hairs.
To compare with hairs shaving or chemical waxing, this new invented device will wax away hairs at the fastest race remove hairs of all.
It is the most economical potent hair removing cosmetically device on the present market.
It is a pain free hairs waxing service.
Within or without waxing to remove hair, the device is also a skin massager.

### Descriptions

The Pain Free Electrical Waxing Body & Legs Hairs Remover is just a small handheld device that is very convenience of using for removing hair of body or legs.
The device can be used to wax away hair any where with home electric available.
This device is an individual cosmetic care.
The design of this new invented electrical waxing hair remover has a smaller scale model that fit work on any limited area of the body.

### DRAWING FIGURES

FIG. 1: This figure shows the drawings of a skin's striking element hitter. It is welded on a turning shaft. Any time this shaft makes a turn, the hitter will hit a skin's striking element once.

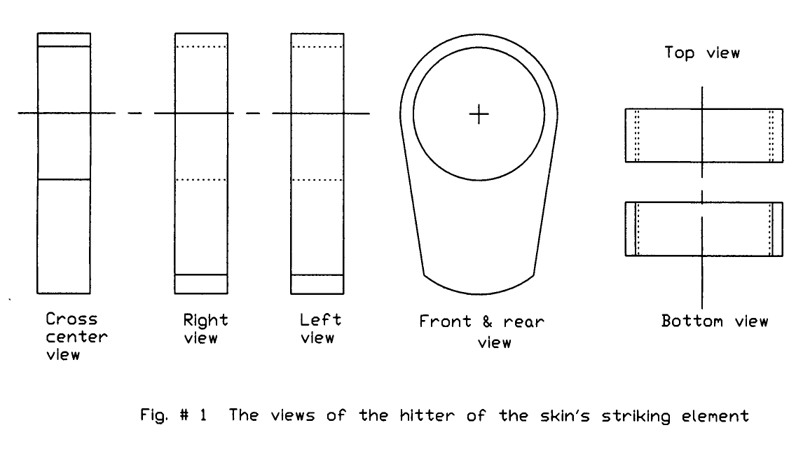

FIG. 2: The drawings show the tweezing element pusher. This pusher is welded on the same turning shaft of skin's striking hitter. Any time the shaft makes a turn, this pusher will move the tweezing element opened and closed by the cone spring for catching and holding hairs.

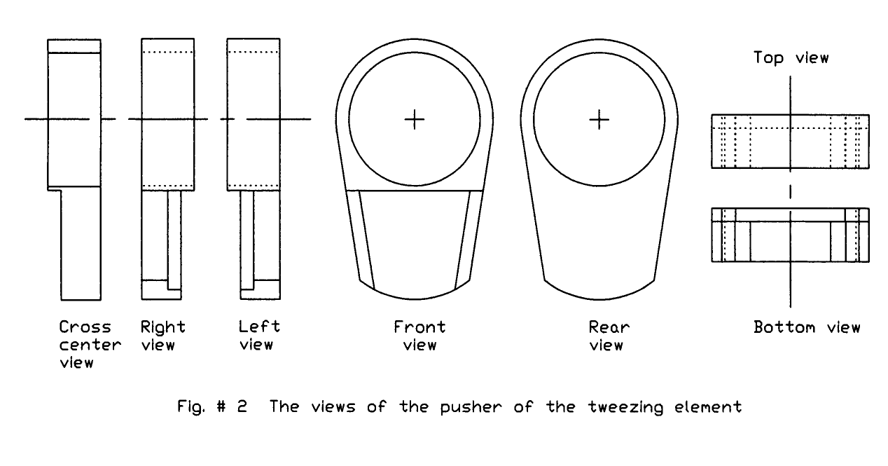

FIG. 3: The drawings show the skin's striking element. This element stays hanging loose on a smaller fixed shaft and between two dividers. This striking element once gets hit by the hitter, will strike on the skin's surface and press the skin down, while the tweezers element is holding the hair. Together, these actions of the two elements, will cause hair to get unplugged.

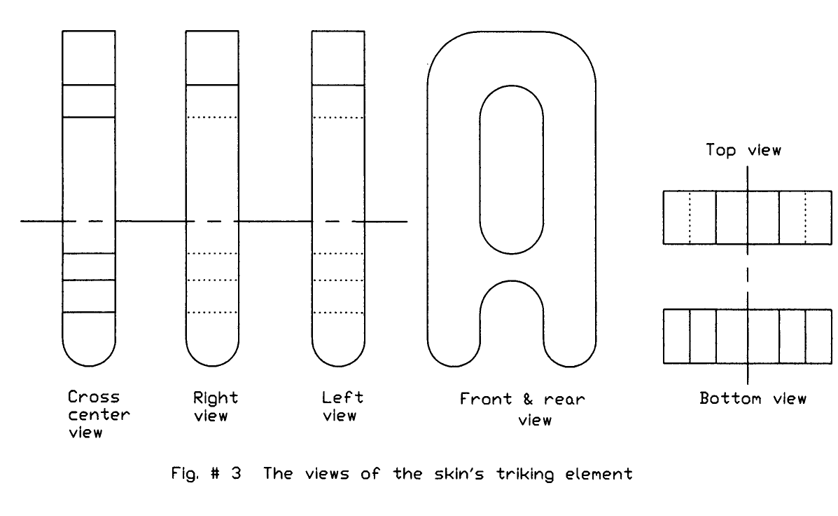

FIG. 4: The drawings show the divider element. There are two of these elements hanging fixed on the same small shaft of striking element and staying guarded on each side of the striking element.

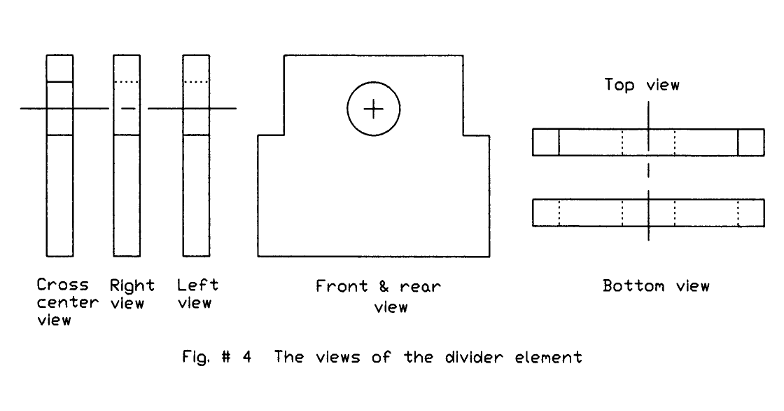

FIG. 5: The drawings show the still tweezing element. This tweezing element is hanging through the same fixed shaft of the skin's striking element. The sides of this element get beveled for hairs easily gathering through.

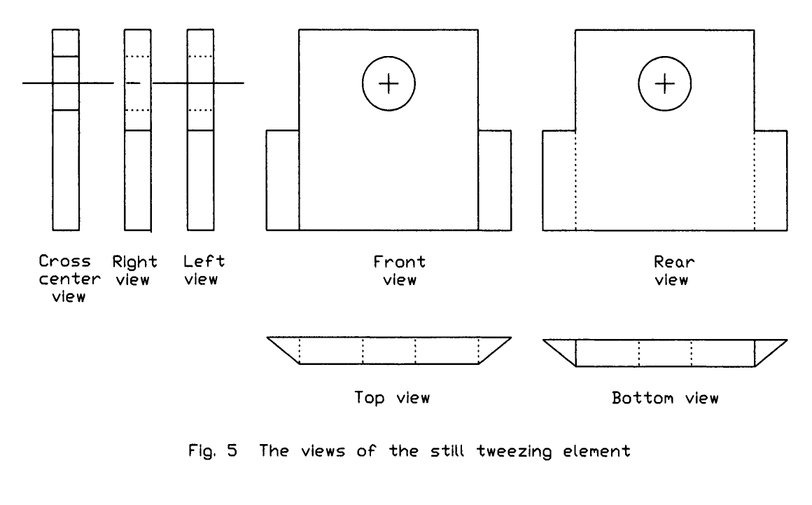

FIG. 6: The drawings show the motion tweezing element. This motion tweezing element is hanging and traveling on the same fixed shaft of the still tweezing element. This motion tweezing element gets pushed to open by the tweezing pusher and closed by a reaction of cone spring on the same shaft. The sides of this tweezing element get beveled for hair easily gathering through.

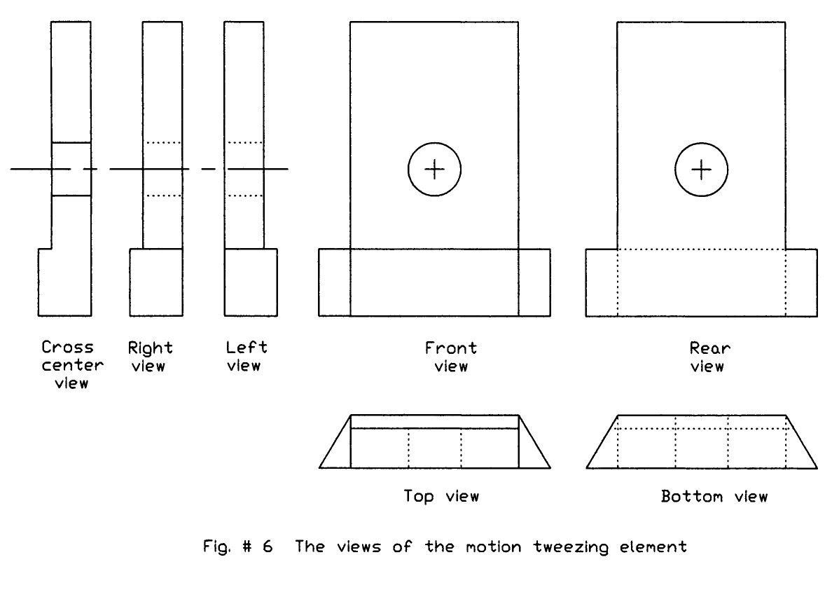

FIG. 7: The figure shows the assembly of elements on the two shafts. The two tweezing pusher and striking hitter are welded on a turning shaft. The skin's striking and tweezers elements are hanging lose on a same smaller fixed shaft. The tweezing elements are hanging between the pair of skin striking elements. There is a cone spring hanging behind the motion tweezing element for forcing the tweezing element to close.

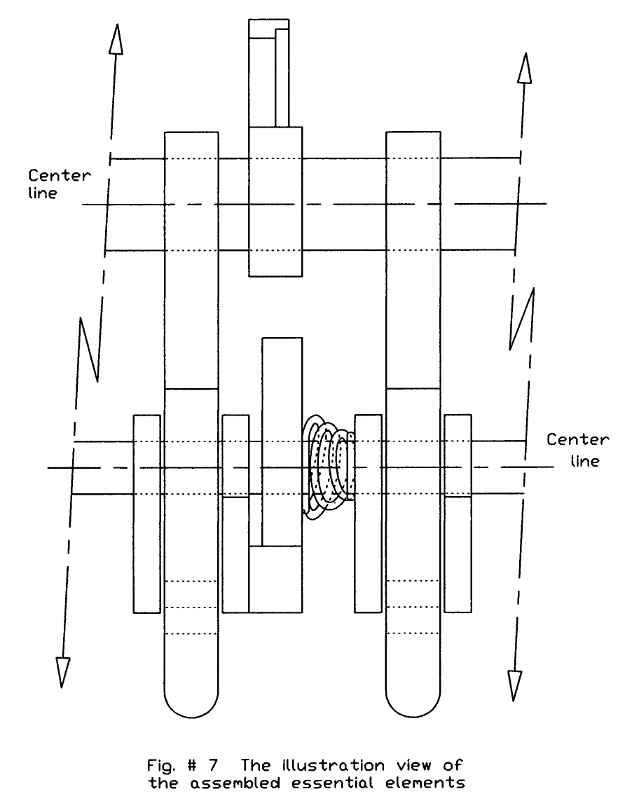

FIG. 8: The figure shows a cross section view of the assembled elements. This figure is to clarify position of elements on the shafts.

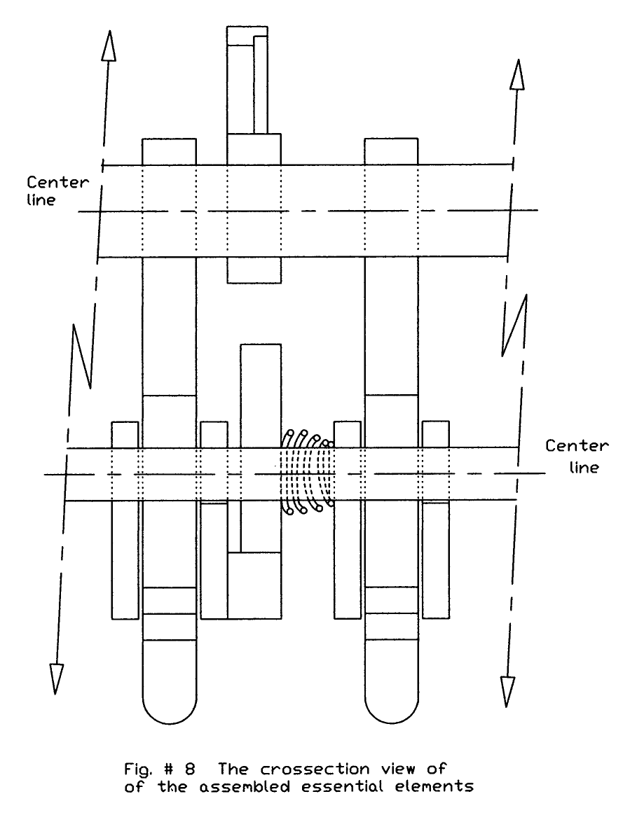

FIG. 9: This figure shows the after match of a half round turning of the shaft. The motion tweezing element is being pushed back. The skin striking element is at the free position while the tweezing element is opening waiting to catch hairs.

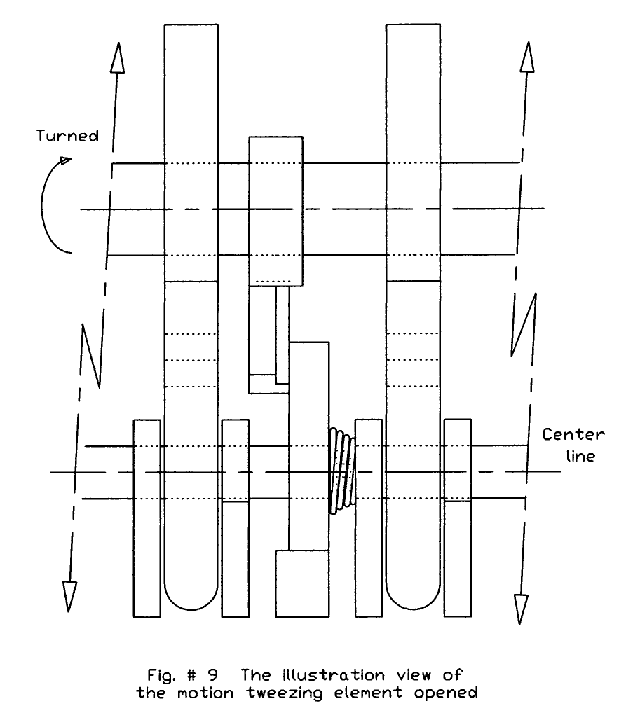

When the turning shaft makes a next half turns, while the tweezers elements are still holding hair, the skin striking element after gets hit from its hitter then launching itself down to strike the skin, the hair then gets to come off.

FIG. 10: The figure shows a true scale of the “Pain Free Electrical Waxing Hair Remover”. The more adds on of tweezers components are the better number of hairs to be removed. The hairs will be pushed out by a rear end fan after being cut.

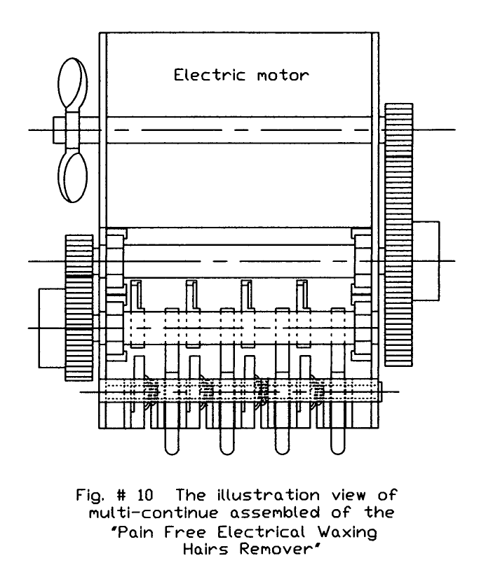

### CONCLUSION

There are many methods and cosmetic products of removing hairs on the market. In the same sense of providing consumer easy way of life, this device is providing a better service, higher quality, lesser pollution of waxing removing hair. The best feature of all is the “Pain Free” in waxing hair away. I believe in making a better society. This new invention will bring some satisfaction to the consumer.

1. The “Pain Free Electrical Waxing Hairs Remover” will wax away hair in a large number of hairs with a minimal time.

2. This new invention device is waxing hairs with out using any chemical agent.

3. This new invention device will wax away hairs with out skin's pain there after.

4. This device has many continuous tweezing components to hold hairs, while an other continuous components press the skin down and strike the skin to get the hairs unplugged. The theorem of this device is: A skin massager that would wax away its hairs.

5. This new invention device is a skin massager, stimulator that helps skin to get normal quickly there after the hairs waxing finished.

6. This new invention of device has a scale down of the size model that fit to remove hairs in some limited area of body. This small unit of device is able to remove hairs of smaller part of body like eyes brown, under arms, mustache, and beard.

7. This new invention device has a dual function: An electrical skin's massager that waxing hairs away at the same time.
